(19) United States
(12) Patent Application Publication
(10) Pub. No.: US 2015/0292024 A1
(43) Pub. Date: Oct. 15, 2015

(54) BIOMARKERS FOR BREAST CANCER PREDICTION AND DIAGNOSIS

(71) Applicants: Tao Wang, Suzhou (CN); Xiangyun Qu, Suzhou (CN); Fei Chen, Suzhou (CN)

(72) Inventors: Tao Wang, Suzhou (CN); Xiangyun Qu, Suzhou (CN); Fei Chen, Suzhou (CN)

(73) Assignee: Suzhou Microdiag Biomedicine Co., Ltd, Suzhou, Jiangsu (CN)

(21) Appl. No.: 14/371,083

(22) PCT Filed: Jan. 9, 2012
(86) PCT No.: PCT/CN2012/070147

§ 371 Date: Nov. 24, 2014

(51) Int. Cl. C12Q 1/68; G01N 33/574

(52) U.S. Cl. CPC C12Q 1/6886; G01N 33/57415; C12Q 2600/158; G01N 2333/8139

(57) ABSTRACT

This invention provides the applications of CST1 gene and its splice variants (sequence shown in SEQ ID No.48) and cystatin SN protein coded by CST1 gene (sequence shown in SEQ ID No.52) for the predictions and diagnoses of human breast cancers. The invention can be used for the diagnosis of breast cancer and breast cancer metastasis, for the analysis for the susceptibility for breast cancer, for the evaluation of the treatments, medicine, efficacy and prognosis for original or metastatic breast cancer patients, and for the risk assessment for breast cancer or its metastasis. The methods described in this invention are of significant sensitivity, specificity and reliability. The invention provides the capturers of CST1 gene, its splice variants and cystatin SN and their applications in manufacturing diagnostic reagents, kits and chips for breast cancers. The diagnostic reagents, kits and chips for breast cancer are included in this invention.

### TECHNICAL FIELD

The present invention is of biomedical technology. A series of biomarkers for cancer prediction and diagnosis, as well as reagents, testing kits and chips for the detections of the biomarkers, is included in the invention.

### BACKGROUND

According to WHO report, around 1.2 million women are diagnosed with breast cancer annually, and 540 thousand deaths related to breast cancers are reported every year. The incidence of breast cancer increases significantly recently. Therefore, it is urgent to develop methods for breast cancer preventions and treatments. Among these methods are early detections and interventions of breast cancer, assessment of cancer treatments and monitoring of the metastases and recurrences of the tumors. One of the criteria to improve breast cancer screening efficiency and post-treatment prognosis monitoring is to develop the diagnostic reagents of high sensitivity and specificity for breast cancer.

Recent investigations of the mechanisms of tumor emergence revealed that cystatin superfamily plays important roles in the occurrence, development, infiltration and metastasis of tumors as cystein protease inhibitors. The expression of several members of cystatin superfamily increases in tumors. For example, cystatin C is relatively over-expressed in ovarian and head and neck tumors. Another example is that the high expression of stefin A in non-small cell lung cancer (NSCLC) cells. The increased expression of cystatin F in tumors is possibly due to the participation of cathepsin in the occurrence and prolification of tumors, during which cathepsin expression rises, leading to the increase of the expression of cystatin to inhibit the overactivity of cathepsin through a feedback mechanism. It's noted however that the expression of cystatin is not always correlated with tumor growth positively. For instance, low expression of cystatin C indicates late stage, poor prognosis and high metastasis possibility among glioma patients. The possible reason lies in some unknown mechanism to regulate cystatin expression at late stage of cancers.

Cystatin SN is a member of human cystatin superfamily. It's coded by CST1 gene and contains 141 amino acid residues. The protein contains two disulfide bonds, and its molecular weight is 16.4 kDa. Cystatin SN is a typical secreted protein, with distributions in body fluids and secretions such as tear, saliva, serum and plasma. It's reported in literature that CST1 expression in gastric cancer cells is higher than in normal gastric cells. The physical distribution of the CST1 expression in gastric cancer cell lines in consistent with the tumor cells. The CST1 expression has a positive correlation with the degree of the differentiation of the cell lines. Preliminary clinical data evidenced that CST1 expression is dependent on tumor infiltration, metastasis status and pTNM staging. Survival analysis indicates that the 5-year overall survival is significantly higher for patients with CST1 expression than those without CST1 expression. Cox analysis shows that CST1 is an independent index for prognosis prediction. All these investigations evidence that CST1 performs as a possible tumor inhibitor during the occurrence and development of gastric cancers. No correlation between CST1 and breast cancer has been reported so far however.

### DETAILED DESCRIPTION

This invention aims at the expression of CST1 of breast cancer patients. Through the analysis of the correlation between CST1 expression and clinical pathological parameters, the role of CST1 in the occurrence, development and metastasis of breast cancer should be revealed.

First, CST1 expression in human tissues were compared in this invention. It was discovered that CST1 expression is low in all tissues tested except the salivary glands. Expression of CST124, CST1, CST2, CST4 in breast cancer tumor and tumor adjacent tissues were compared. It was discovered that CST1 expression has the largest difference in tumor and adjacent tissues. CST1 mRNA (SEQ ID No.44) has two splice variants: splice 1 (SEQ ID No.48) contains exon 1 (SEQ ID No.45), exon 2 (SEQ ID No.46) and exon 3 (SEQ ID No.47); splice 2 (SEQ ID No.49) contains only exon 1 (SEQ ID No.45) and exon 2 (SEQ ID No.46). We compared the expression difference of these two variants in tumors and tumor adjacent tissues in this invention. We discovered that splice 1 has larger difference than splice 2. The expressions of splice 1 of CST1 were compared in breast cancer tumors, tumor adjacent tissues, biopsy samples of breast cancerous tumors, mastitis and normal tissues, lymph node tissues with and without breast cancer metastasis, cell-free RNA of breast cancer patients, mastitis patients and health women. It was discovered that splice 1 of CST1 is over expressed in breast cancer tumors and tissues with breast cancer metastasis. It was realized to differentiate breast cancer tumors, tumor adjacent tissues, normal tissues and tissues with or without breast cancer metastasis with high sensitivity and specificity via quantitative measurement of the expression splice 1 of CST1.

Second, this invention compared the expression of cystatin SN (sequence presented in SEQ ID No.52, gene code presented in SEQ ID No.53) in breast cancer cell lines and normal serums, in serums of breast cancer patients and healthy people. It was discovered that cystatin SN is over expressed in breast cancer cell lines and breast cancer patients serums. Through quantitative measurement of cystatin SN expression, it was realized to distinguish breast cancer tumors and breast cancer patients serums from normal serums with high sensitivity and specificity,

Combining the mentioned discoveries, it is concluded that splice 1 of CST1 and cystatin SN can be used as biomarkers for the prediction and diagnosis of breast cancers. The epitope peptide of cystatin SN (cystatin SN sequence after the removal of the signal peptide sequences) can be also applied as a biomarker for prediction and diagnosis of breast cancers as a conclusion of clinical common sense. The sequence of the epitope peptide of cystatin SN is presented in SEQ ID No.54.

Breast cancers can be predicted and diagnosed via measuring the expression of the biomarkers mentioned above. Applications include the discrimination and diagnosis of original and metastatic breast cancers, susceptibility analysis for breast cancer, efficacy evaluations for the treatments and medicine of original or metastatic breast cancer, and risk assessment for breast cancer or its metastasis, etc. For example, one of the methods can be described as following: the expression of splice 1 of CST1 or cystatin SN is measured and compared with a cutoff value. The conclusion of the test is positive if the expression of the biomark(s) is higher than the cutoff value. The cutoff value is obtained by counting and comparing the expression or content of splice 1 of CST1 or cystatin SN of body fluids or breast tissues of healthy people and breast cancer patients.

Testing reagents, kits and chips are required for measuring the expression of the biomarkers mentioned. Therefore, these reagents, kits and chips for breast cancer predictions and diagnoses are investigated in this invention.

The invention includes multiple methods for mRNA detections, which include, but not limited to polymerase chain reaction (PCR), nucleic acid based amplification (NASBA), transcription-median amplification (TMA), Ligase chain reaction (LCR) and thermophilic strand displacement amplification (tSDA). Primers or probes that specifically recognize splice 1 of CST1 or its cDNA (capturer of splice 1 of CST1) and match the requirements of the detection methods can be used to manufacture testing reagents for prediction and diagnosis of breast cancer. Primers and probes that recognize exon 1, 2 and 3 are applied in this invention, among which exon 1-specific primers and probes are preferred. For example, the sequence of the primers that can be used in this invention should match at least one of what are presented in SEQ ID No.1-2, 4-21, 34 and 39-42. Sequences shown in SEQ ID No.1-2 are of high favor. Sequences of applicable probes should match at least one of what are presented in SEQ ID No.3, 35-38 and 43, while sequence shown in SEQ ID No.3 is preferred.

The invention includes multiple methods for protein detections, which include, but not limited to Enzyme-linked immunosorbent assay (ELISA) and its derivatives such competitive ELISA, double-antibody sandwich ELISA, immunoblotting, ELISA-immunoblotting, etc. Antibodies that recognize cystatin SN (capturers of cystatin SN) can be used in testing reagents for prediction and diagnosis of breast cancer. The antibodies mentioned include monoclonal or polyclonal antibodies, and are able to be labeled with enzyme such as alkaline phosphatase (ALP), luciferase, oxidase, β-galactosidase and various fluorophores. The antibodies can be biotin labeled and the signal is amplified by streptavidin-substrate complex. Quantification is realized through the UV absorbance change upon the interaction of the substrate and the antibody labeled enzyme; or it's realized by UV excitation of the fluorophore-labeled antibodies.

The testing reagents might be combined with each other or with other auxiliary testing reagents to make testing kits for the prediction and diagnosis of breast cancer for convenient and rapid testing. Any commercial or literature-reported testing kits that contain the testing reagents mentioned above might also be applied for the diagnosis and prediction of breast cancers.

As an optimized technology, testing kits for splice 1 of CST1 based on real time PCR with TaqMan probes contains at least one pair of primers and one probe that specifically recognize the analyte. Primers' sequences are presented in SEQ ID No.1-2 and probe's sequence is shown in SEQ ID No.3 with fluorophore and fluorescent quencher labeling on the 5′ and 3′ terminus respectively.

As an optimized technology, testing kits for splice 1 of CST1 based on real time PCR with fluorophores contains at least one pair of primers. The capturers of splices of CST1 are primers that specifically recognize cDNA of the splices of CST1. Sequences of the primer pairs are presented in SEQ ID No.1-2, or SEQ ID No.4-5, or SEQ ID No.6-7, or SEQ ID No.8-9, or SEQ ID No.10-11, or SEQ ID No.12-13, or SEQ ID No.14-15, or SEQ ID No.16-17, or SEQ ID No.18-19, or SEQ ID No.20-21.

As an optimized technology, testing kits for splice 1 of CST1 based on NASBA or TMA contains at least one pair of primers and one probe that specifically recognize the cDNA of splice 1 of CST1. Primers sequences are shown in SEQ ID No.34 and SEQ ID No.2. The probe sequence is shown in SEQ ID No.3, with fluorophore and fluorescent quencher labeling on the 5′ and 3′ terminus respectively.

As an optimized technology, testing kits for splice 1 of CST1 based on LCR contains four probes that specifically recognize the analyte. Their sequences are presented in SEQ ID No.35-40 with hapten labeling on the 5′ termini.

As an optimized technology, testing kits for splice 1 of CST1 based on tSDA contains at least one pair of the primers and one probe that specifically recognize the cDNA of splice 1 of CST1. Primers' sequences are shown in SEQ ID No.39-44 and probe's sequence is shown in SEQ ID No.43 with radioactive labeling on the 5′ terminus.

One or more auxiliary reagents can be added to all testing kits for splice 1 of CST1 mentioned above. These auxiliary reagents include, but not limited to 1) reagents that make the respective amplicons of the primers visible or imageable. The methods include agarose gel electrophoresis, enzyme-linked gel electrophoresis, chemoluminescence, fluorescent in situ hybridization (FISH) and fluorescent microscopy. 2) RNA extraction reagents. 3) Reverse transcription reagents. 4) cDNA amplification reagents such as reagents applied in PCR, quantitative PCR (qPCR), NASBA, TMA, LCR and tSDA. 5) Standards for calibrations such as recombinant plasmid that contains splice 1 of CST1. 6) Positive control such as human breast cancer cell lines HCC1937, SK-BR-3 and MCF-7. 7) Negative control such as normal breast tissue cell lines Hs578Bst.

As an optimized technology, testing kits for cystatin SN based on competitive ELISA contains cystatin antigen, anti-cystatin SN monoclonal antibody, enzymatic labeled secondary antibody and the substrate of the enzyme. In a typical process, the ELISA plate is coated by cystatin SN antigen. Samples and anti-cystatin SN antibodies are incubated on the plate successively. Cystatin SN in samples is quantitatively decided by measuring the signal of the reporting reagents (enzymatic labeled secondary antibodies and its corresponding substrate).

As an optimized technology, testing kits for cystatin SN based on double-antibody sandwich ELISA contains cystatin antigen, anti-cystatin SN monoclonal antibody, biotin-labeled anti-cystatin SN polyclonal antibody, streptavidin-enzyme complex and substrate. In a typical process, the ELISA plate is covered by cystatin SN antigen. Samples and biotin-labeled anti-cystatin SN polyclonal antibodies are incubated on the plate successively. Cystatin SN in samples is quantitatively decided by measuring the signal of the reporting reagents (streptavidin-enzyme complex and its corresponding substrate).

One or more auxiliary reagents can be added to all testing kits for cystatin SN mentioned above. These auxiliary reagents include, but not limited to 1) ELISA coating solution. 2) Antibody dilution buffer. 3) Washing buffer. 4) Reaction stop solution. 5) Cystatin SN standard for calibration.

Probes for the detections of splice 1 of CST1 or its cDNA can be immobilized on solid substrate to make a sensing chip for breast cancer predictions and diagnoses aiming at convenient and rapid testing. Biochips reported in literatures or commercially available chips that contain the probes mentioned above can also be used to make sensing chips for the prediction and diagnosis of breast cancer.

Testing reagents, kits and chips mentioned in this invention can be used to test the susceptibility for breast cancer gaining, to decide pTNM stage of breast cancer, to evaluate the development of the cancer and the efficacy of the treatments, and to detect the recurrence and metastasis of the breast cancer. Samples include surgical or biopsy tissues, lymph node tissues, marrow, serum, plasma, whole blood, fractions of blood samples and urine. Testees are people who seek medical assistance due to breast discomfort, who have family history of breast cancers and who have breast cancer.

The benefits of this invention lie in the following facts. The sequences of splice 1 of CST1 and its coded protein cystatin SN, shown in SEQ ID No.48 and 52 respectively, are published as biomarkers for the prediction and diagnosis of breast cancers. Via measuring the expression of these biomarkers, one can distinguish and diagnose the occurrence or metastasis of breast cancers, test the susceptibility of breast cancer gaining, evaluate the efficacies of the treatments for original and metastatic breast cancer, predict the cancer prognosis and assess the risk of people to acquire breast cancer or its metastasis. The methods described in this invention are of high sensitivity, specificity and reliability. The capturers for the splices of CST1 and cystatin SN are published in this invention, as well as their application in manufacturing the testing reagents, kits and chips for breast cancer predictions and diagnoses.

### BRIEF DESCRIPTION OF THE FIGURES

FIG. 1. Recombinant plasmid that contains splice 1 of CST1 (pMD18-T-CST1)

FIG. 2. Expression of CST1 gene in normal tissues (tonsil, posterior pituitary, thyroid, salivary gland, skeletal muscle, bone marrow, peripheral blood without red blood cells and platelets, lung, stomach, liver, heart, kidney, adrenal gland, intestines, colon, pancreas, spleen, bladder, prostate, breast, ovarian, uterus, placenta and testis), human breast cancer cell lines (HCC1937, SK-BR-3, RI MCF-7) and normal breast tissue cell line (Hs578Bst)

FIG. 3. Expression comparison of CST124, CST1, CST2, CST4 in breast cancer tumors and tumor adjacent tissues using real time qPCR with fluorescent dye as the probe.

FIG. 4. Expression comparison of ACTB in breast cancer tumors and tumor adjacent tissues using real time qPCR with fluorescent dye as the probe. N1-N20 and T1-T20 correspond to breast cancer tumor samples and tumor adjacent tissues respectively.

FIG. 5. Expression comparison of splice 1 and splice 2 of CST1 in breast cancer tumors and tumor adjacent tissues using real time qPCR with fluorescent dye as the probe.

FIG. 6. Quantification through real time PCR of splice 1 of CST1 expression in breast cancer tumors, tumor adjacent tissues and normal tissues

FIG. 7. Quantification through real time PCR of splice 1 of CST1 expression in breast cancer tumors and biopsy samples with mastitis

FIG. 8. Quantification through real time PCR of splice 1 of CST1 expression in lymph node tissues with and without breast cancer metastasis

FIG. 9. Quantification through real time PCR of splice 1 of CST1 expression in cell-free RNA of patients with breast cancers and mastitis, and healthy people.\

FIG. 10. Receiver operator characteristic curve (ROC) of the test, which indicates that splice 1 of CST1 expression has high sensitivity and selectivity as a marker for breast cancer diagnosis

FIG. 11. Expression difference through LCR of splice 1 of CST1 in cell-free RNA in the serums of breast cancer and mastitis patients and healthy people

FIG. 12. Expression difference through tSDA of splice 1 of CST1 in cell-free RNA in the serums of breast cancer and mastitis patients and healthy people

FIG. 13. Expression difference through NASBA of splice 1 of CST1 in cell-free RNA in the serums of breast cancer and mastitis patients and healthy people

FIG. 14. Comparison of the sensitivities of the detections of micrometastasis of breast cancer by splice 1 of CST1 expression in peripheral blood and by cytological analysis

FIG. 15. Comparison of the sensitivities of the detections of micrometastasis of breast cancer by splice 1 of CST1 expression in bone marrow and by cytological analysis

FIG. 16. Expression difference through TMA of splice 1 of CST1 in cell-free RNA in the serums of breast cancer patients with different TNM staging

FIG. 17. Expression of cystatin SN in breast cancer tumor cell lines and serums of healthy people

FIG. 18. Expression of cystatin SN in serums of breast cancer patients and healthy people

FIG. 19. Expression of cystatin SN in serums of breast cancer patients and healthy people measured by competitive ELISA

FIG. 20. Expression of cystatin SN in serums of breast cancer patients and healthy people measured by double-antibody sandwich ELISA

FIG. 21. Specificity and sensitivity comprison of cystatin SN and CEA in breast cancer patients serums. Protein concentrations were measured by ELISA

FIG. 22. Survival rate of post-treatment breast cancer patients with two groups: cystatin SN expression higher than the median value and lower than the median value

### EXAMPLES

Several detailed descriptions of examples for the applications of this invention are provided in the following texts. Figures apply when necessary. Experimental details for routine molecular biology procedures mentioned in these examples are not included. Protocols in Molecular Cloning: A Laboratory Manual (3^{rd} edition, Edited by J. Sambrook et al) were strictly followed for these procedures. Protocols in the user manuals of the bioreagents are observed in our experiments. If not clarified, all percentages mentioned in these examples are percentages by weight (w.t. %).

### Nucleic Acid

All samples in the examples are acquired after the reception of patients signed consent forms; Regulations of the medical institutes where the samples were acquired were strictly observed. Biopsy samples of tumor tissues were compared with normal tissues. RNA extraction follows the acquisition of the lymph node samples from surgery immediately, or the samples were stored in liquid nitrogen or RNAlater (Ambion). Peripheral blood, marrow or urine samples were centrifuged for 20 minutes (4000 rpm, 4). The supernatants were centrifuged for another 10 minutes (13000 rpm, 4). RNA extraction followed immediately or the samples were stored under low temperature (−20-−80).

### Example 1

### CST1 Expressions in Human Tissues

All normal tissues in this example were acquired from medical institutes in collaboration with the inventors. Other samples were purchased commercially and corresponding laws and regulations were observed. CST1 mRNA expression in various human tissue samples was measured on HG-U95AV Human GeneChip Array (Affymetirx); protocols on the user manual are followed. Quantifications of CST1 mRNA expressions were realized by the β-actin fluorescence calibration curve. The results are presented in FIG. 2. CST1 is not expressed in normal tissues except the saliva glands. This discovery evidences that CST1 matches one of the requirements of a good diagnostic biomarker—low background in normal tissues. Results in FIG. 2 also points that CST1 is over expressed in breast cancer tumor cell lines HCC1937, SK-BR-3 and MCF-7, and not expressed in normal breast tissue cell line Hs578Bst. The results indicate that CST1 is a potential biomarker for breast cancers.

### Example 2

### Expressions of CST124, CST1, CST2 and CST4 in Breast Cancer Tumors and Tumor Adjacent Tissues

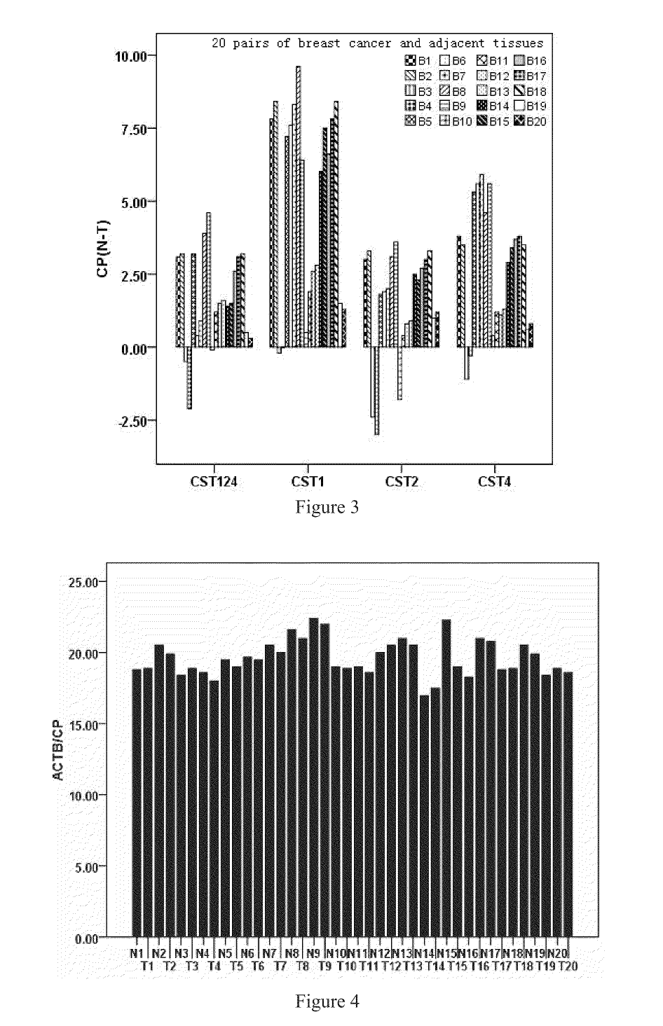

Real time qPCR with fluorescent dye as the probe was used to measure the mRNA expressions of CST124, CST1, CST2 and CST4 in 20 breast cancer tumor samples and their respective tumor adjacent tissues. Trizol reagents were used for RNA extractions. The reverse transcriptions of the mRNA were realized using commercially available kits with the user manual followed. Primers for the PCR amplifications of cDNA are summarized in Table 1. The fluorescent dyes are SYBR Green, Eve Green and LC Green etc. As shown in FIG. 3, CST1 has the largest expression difference in tumors and tumor adjacent tissues among all the genes tested.

TABLE 1
PCR primers for CST124, CST1, CST2 and CST4

| Gene | Primer 1(5′→3′) | Primer 2(5′→3′) |
|---|---|---|
| CST124 | agtcccagcccaacttgg | gggaacttcgtagatctgga |
| | a (SEQ ID No. 24) | aaga (SEQ ID No. 25) |
| Splice 1 | tctcaccctcctctcctg | ttatcctatcctcctccttg |
| of CST1 | (SEQ ID No. 1) | g (SEQ ID No. 2) |
| Splice2 | gcactggggaagagaggc | aggagcagcagggtactcag |
| of CST1 | at (SEQ ID No. 26) | ata (SEQ ID No. 27) |
| CST2 | cagaagaaacagttgtgc | ggagtaggaggtggtcag |
| | tc (SEQ ID No. 28) | (SEQ ID No. 29) |
| CST4 | gctctcaccctcctctcc | tatcctattctcctccttgg |
| | tg (SEQ ID No. 30) | (SEQ ID No. 31) |
| β-actin | aagatcattgctcctcct | cgtcatactcctgcttgc |
| reference | g (SEQ ID No. 32) | (SEQ ID No. 33) |

### Example 3

### Expressions of Splices 1 and 2 of CST1 in Breast Cancer Tumors and Tumor Adjacent Tissues

Real time qPCR with fluorescent dye as the probe was used to measure the mRNA expressions of ACTB gene in 20 breast cancer tumor samples and their respective tumor adjacent tissues. As shown in FIG. 4, the cross point values (CP) for ACTB gene are close. Next, Real time qPCR with fluorescent dye as the probe was used to measure the mRNA expressions of both slices of CST1 in 20 breast cancer tumor samples and their respective tumor adjacent tissues. The protocol and primers design for PCR were identical to those in Example 2. As shown in FIG. 5, splice 1 has the larger expression difference in tumors and tumor adjacent tissues than splice 2, which indicates that splice 1 is advantageous than splice 2 as a diagnostic biomarker for breast cancer.

### Example 4

### Expressions of Splices 1 of CST1 in Breast Cancer Tumors and Tumor Adjacent Tissues and Normal Tissues

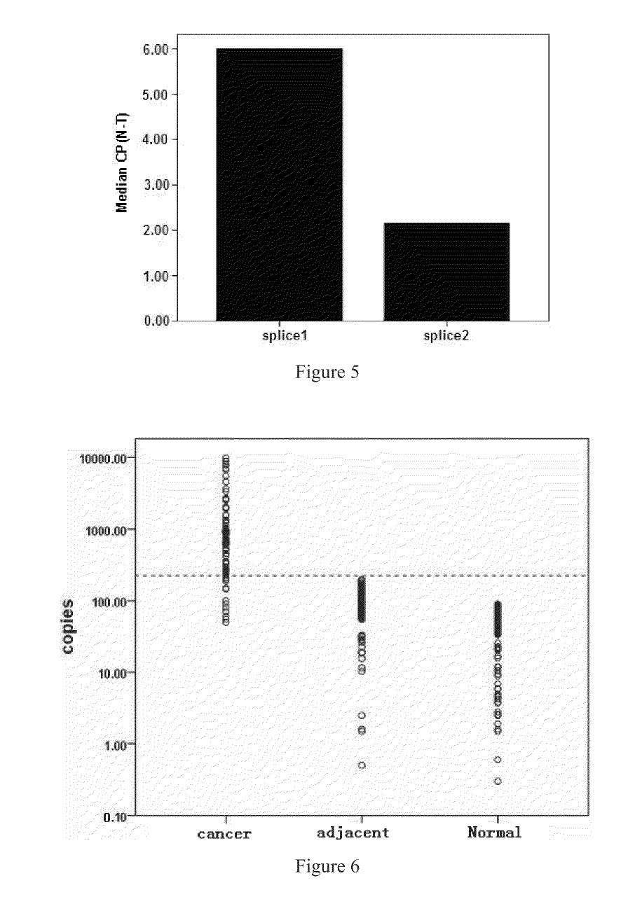

Expressions of splice 1 of CST1 in breast cancer tumors, tumor adjacent tissues and normal tissues were quantified by real time PCR. The sequences of the PCR primers are presented in SEQ ID No.1 and SEQ ID No.2. The amplicon sequence is shown in SEQ ID No.50. As shown in FIG. 6, splice 1 of CST1 is over expressed in breast cancer tumor tissues, while its expression is relatively low in tumor adjacent tissues and normal tissues. Median of splice 1 expression in tumors are 15.6 fold higher than that of normal tissues. Cancerous tumors can be distinguished from normal tissues if 223.20 is used as the cutoff value, which is proposed as a reference for breast cancer diagnosis.

### Example 5

### Expressions of Splices 1 of CST1 in Biopsy Samples of Patients with Breast Cancer and Mastitis

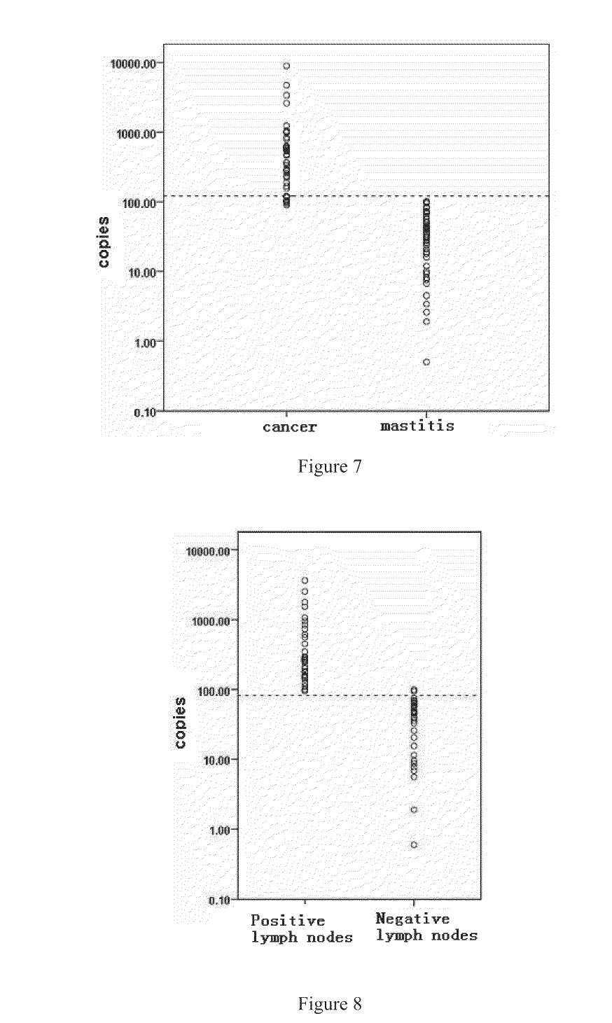

Samples from surgery and biopsy are of significant difference in that the percentage of cancer cells in biopsy samples varies. Thus, 40 biopsy samples from patients with breast cancer or mastitis were tested using real time PCR. Sequences for PCR primers are shown in SEQ ID No.1-2. As shown in FIG. 7, expression of splice 1 of CST1 is higher in breast cancer samples than in mastitis samples. The median of breast cancer CST1 expression is 11.0 fold higher than that of mastitis. Cancerous tumors can be distinguished from tissues with mastitis if 120.66 copies is used as the cutoff value, which is proposed as a reference for breast cancer diagnosis.

### Example 6

### Expression of Splice 1 of CST1 in Lymph Node Samples with and without Breast Cancer Metastasis

We collected 30 lymph node samples with breast cancer metastasis of different sizes. 30 lymph node samples without breast cancer metastasis are from early stage breast cancer patients to avoid undetectable lymph node metastasis. Real time PCR was applied for the quantification of the expressions of splice 1 of CST1 in these lymph node samples. The sequences of the PCR primers are presented in SEQ ID No.1 and SEQ ID No.2. The sequence of the amplicon is presented in SEQ ID No.50. As shown in FIG. 8, the expressions of splice 1 of CST1 in lymph node samples with breast cancer metastasis are higher than those without metastasis. Median for splice 1 expression in metastatic samples is 6.3 fold higher than in samples without metastasis. If 82.45 copies is defined as the cutoff value, 2 cases without breast cancer metastasis are detected splice 1 expression positive. Careful cytological study was performed and very small breast cancer metastasis was discovered in these two samples. The example presents that the expression test of splice 1 of CST1 not only distinguishes metastasis cases with 82.45 copies as the cutoff value, but it is more sensitive than cytological analysis.

### Example 7

### Expression of Splice 1 of CST1 in Cell-Free RNA in Serums of Patients with Breast Cancer, Mastitis and Healthy People

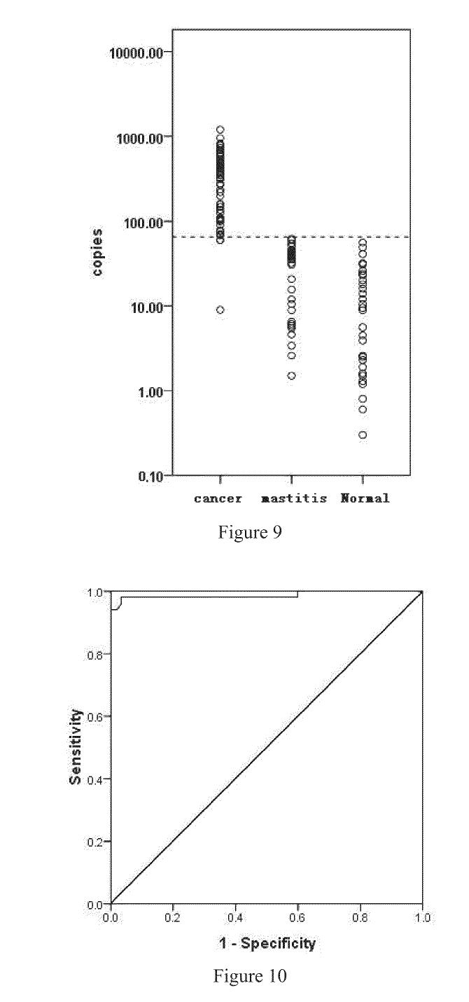

Cell-free RNA was extracted by commercially available kits. Splice 1 of CST1 expressions in cell-free RNA of serums of 50 breast cancer patients, 30 mastitis patients and 30 healthy people were quantified by real time PCR. The sequences of the PCR primers are presented in SEQ ID No.1 and SEQ ID No.2. The sequence of the amplicon is presented in SEQ ID No.50. As shown in FIG. 9, median for splice 1 expression in breast cancer samples is 8.8 fold higher than in samples with mastitis and 32 fold higher than in samples of healthy people. 65.32 copies as the cutoff value can distinguish cancer from inflammation and healthy people. FIG. 10 presents the receiver operating characteristic (ROC) curve of the test, which indicates that splice 1 of CST1 expression has high sensitivity and specificity as a marker for breast cancer diagnosis. The area under the curve (AUC) is 0.987, a value that validates splice 1 of CST1 as a specific marker for non-invasive breast cancer diagnosis.

### Example 8

### Expression of Splice 1 of CST1 in Cell-Free RNA in Serums of Patients with Breast Cancer, Mastitis and Healthy people by LCR

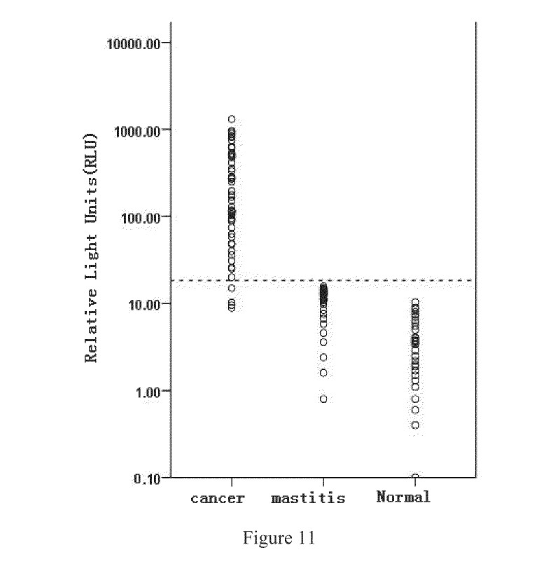

Cell-free RNA was extracted by commercially available kits. Splice 1 of CST1 expressions in cell-free RNA of serums of 50 breast cancer patients, 30 mastitis patients and 30 healthy people were quantified LCR. The sequences of the LCR primers are presented in SEQ ID No.1 and SEQ ID No.2. The sequence of the amplicon is presented in SEQ ID No.50. As shown in FIG. 11, the relative light units (RLU) of the fluorescence of splice 1 of CST1 in cancerous samples is higher than in samples with mastitis and normal samples, with median value 12.38 and 40.12 fold higher, respectively. Cancers are distinguished if 18.51RLU set as the cutoff.

### Example 9

### Expression of Splice 1 of CST1 in Cell-Free RNA in Serums of Patients with Breast Cancer, Mastitis and Healthy People by tSDA

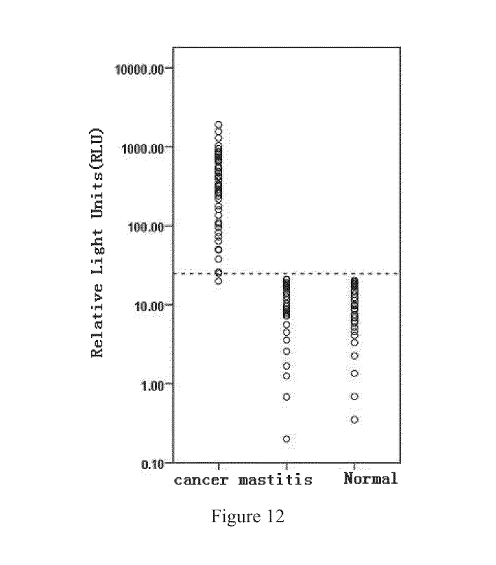

Cell-free RNA was extracted by commercially available kits. Splice 1 of CST1 expressions in cell-free RNA of serums of 50 breast cancer patients, 30 mastitis patients and 30 healthy people were quantified tSDA. The sequences of the tSDA primers are presented in SEQ ID No.1 and SEQ ID No.2. The sequence of the amplicon is presented in SEQ ID No.50. As shown in FIG. 12, the expressions of splice 1 of CST1 in cancerous samples is higher than in samples with mastitis and normal samples, with median value 41.38 and 42.86 fold higher, respectively. Cancers are distinguished if 24.81RLU set as the cutoff.

### Example 10

### Expression of Splice 1 of CST1 in Cell-Free RNA in Serums of Patients with Breast Cancer, Mastitis and Healthy People by NASBA

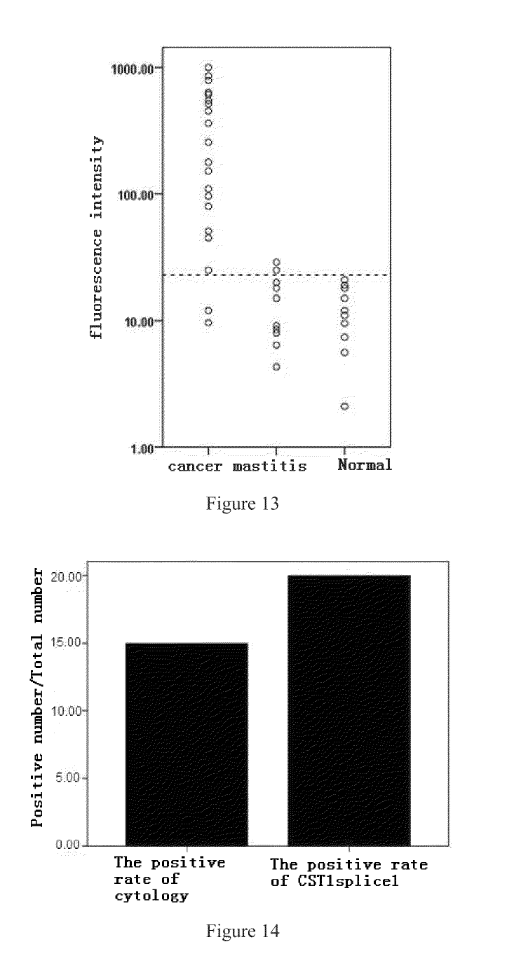

Cell-free RNA was extracted by commercially available kits. Splice 1 of CST1 expressions in cell-free RNA of serums of 50 breast cancer patients, 30 mastitis patients and 30 healthy people were quantified NASBA. The sequences of primers are presented in SEQ ID No.1 and SEQ ID No.2. The sequence of the amplicon is presented in SEQ ID No.50. As shown in FIG. 13, RLU of the fluorescence in cancerous samples is higher than in samples with mastitis and normal samples, with median value 18 and 18.87 fold higher, respectively. Cancers are distinguished if RLU of 22.93 set as the cutoff.

### Example 11

### Splice 1 of CST1 Expression in Breast Cancer Patients Peripheral Blood and its Comparison with Cytology Studies

Red blood cells and platelets were removed from blood of breast cancer patients to make peripheral blood, from which RNA was extracted. The expression of splice 1 of CST1 was quantified by real time PCR. The sequences of primers are presented in SEQ ID No.1 and SEQ ID No.2. The sequence of the amplicon is presented in SEQ ID No.50. The results were compared with the splice 1 expression in blood samples of mastitis patients and healthy people. The high expression of splice 1 is an indication of the existence of cancer cells. And cytology studies was used to verify the cancer cell existence as a gold standard. The results are summarized in FIG. 14. All cytologically diagnosed breast cancers are identified by splice 1 expression testing. Some cases without breast cancer decided by cytology studies are later verified with metastasis. The means splice 1 of CST1 is a more sensitive method for breast cancer diagnosis than cytology.

### Example 12

### Splice 1 of CST1 Expression in Breast Cancer Patients Marrow and its Comparison with Cytology Studies

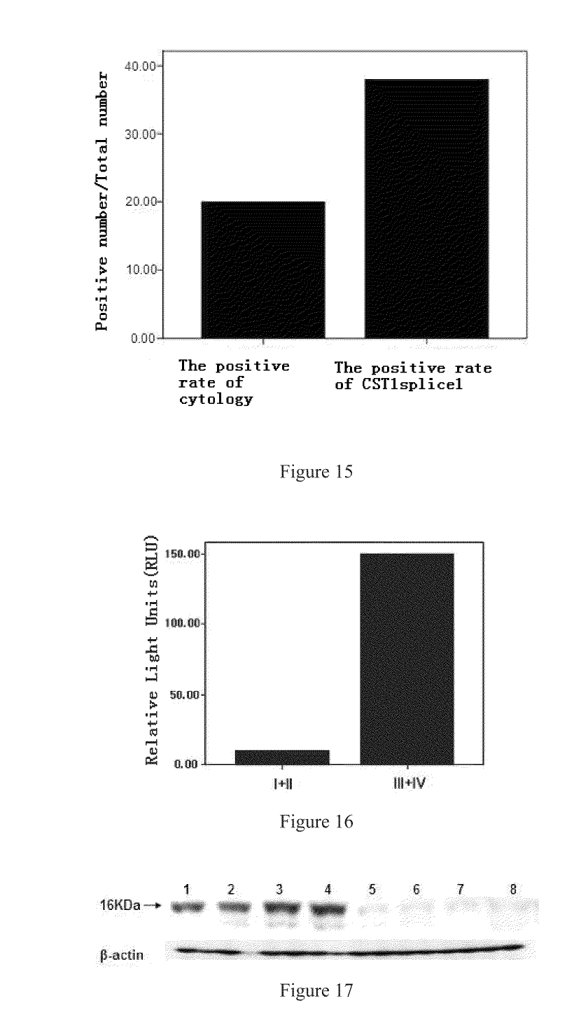

Real time PCR was applied to quantify the expression of splice 1 of CST1 in breast cancer patients marrow samples. The sequences of primers are presented in SEQ ID No.1 and SEQ ID No.2. The sequence of the amplicon is presented in SEQ ID No.50. The results were compared with normal marrow samples. The high expression of splice 1 is an indication of the existence of cancers and cancerous metastasis. And cytology studies was used to verify the cancer cell existence as a gold standard. As shown in FIG. 15, 95% of cytological positive samples were identified by splice 1 of CST1 expression testing. Expression testing has higher positive rate than cytological studies, indicating its higher sensitivity.

### Example 13

### pTNM Staging by Expression of Splice 1 of CST1

Cell-free RNA of 80 breast cancer patients with various pTNM stages (30 cases for I and II, 50 cases for III and IV) was extracted by commercially available kits. TMA was applied to test splice 1 expression. The sequences of primers are presented in SEQ ID No.1 and SEQ ID No.2. The sequence of the amplicon is presented in SEQ ID No.50. As shown in FIG. 16, RLU for samples of stages III and IV is higher than samples of stages I and II, with median value 15 fold higher. The results evidence that expression of splice 1 of CST1 is a marker for pTNM staging.

### Example 14

### Application of the Expression of Splice 1 of CST1 in the Monitoring of Breast Cancer Treatments

Real time PCR was applied for the quantifications of the expression of splice 1 of CST1 in breast cancer patients (6 taking chemotherapy and 4 taking radiotherapy) blood. The sequences of primers are presented in SEQ ID No.1 and SEQ ID No.2. The sequence of the amplicon is presented in SEQ ID No.50. Splice 1 expression was monitored for the evaluation of the treatments. As shown in Table 2, for patients with effective treatments, splice 1 of CST1 expression decreased with the continuation of the therapies, with decreased size of tumors observed by imaging. For patients without effective treatments, splice 1 of CST1 expression increased with the continuation of the therapies, with increased size of tumors observed by imaging. It's proposed that expression of splice 1 of CST1 be applied as a criterion for therapy efficacy and marker for real time monitoring of cancer development.

TABLE 2
Expression of splice 1 of CST1 of breast cancer patients
taking therapies

| | | Expression of splice 1 | | | | | |
|---|---|---|---|---|---|---|---|
| | | of CST1 (copies) | | | Tumor size (cm) | | |
| | | Cycle | Cycle | Cycle | Cycle | Cycle | Cycle |
| | Therapies | 1 | 2 | 3 | 1 | 2 | 3 |
| Effective | Chemo2 | 1000.8 | 412.3 | 80.5 | 2.5 | 1.5 | <1 |
| treatment | Chemo5 | 5632.6 | 1213.6 | 110.5 | 3.5 | 2.5 | <1 |
| | Chemo6 | 1316.5 | 564.5 | 50.8 | 3 | 2 | <1 |
| | Radio1 | 1890.6 | 1060.9 | 156.8 | 3 | 1.5 | 1 |
| | Radio4 | 2562.6 | 1589.4 | 200.6 | 3 | 2.5 | 1.5 |
| Ineffective | Chemo1 | 1568.6 | 1984.6 | 2530.5 | 3 | 3 | 3.5 |
| treatment | Chemo3 | 856.9 | 1056.3 | 1646.2 | 2 | 2 | 2.5 |
| | Chemo4 | 543.2 | 846.7 | 1132.5 | 1 | 1 | 1.5 |
| | Radio2 | 1132.4 | 1346.8 | 1889.6 | 2 | 2.5 | 3 |
| | Radio3 | 986.4 | 1264 | 1564 | 1 | 1.5 | 2 |

### Example 15

### Prognosis by Expression of Splice 1 of CST1 of Breast Cancer Patients

Expression of splice 1 of CST1 in breast cancer patients blood was quantified by real time PCR. The sequences of primers are presented in SEQ ID No.1 and SEQ ID No.2. The sequence of the amplicon is presented in SEQ ID No.50. The prognosis of cancers were evaluated 1 month, 3 months and one year later than the surgeries. As shown in Table 3, there are 2 cases with cancer recurrence and expression of splice 1 of CST1 increase; the metastasis was confirmed by imaging when the expression of splice 1 of CST1 reached 1000 copies. For the other three cases, no imaging evidence for recurrence or CST1 expression increase were observed. The consistency validates the expression of splice 1 of CST1 as a breast cancer prognosis marker.

TABLE 3
Expression of splice 1 of CST1 in breast cancer patients blood after
chemo- or radiotherapies

| | | Splice 1 of CST1 expression | | | | | |
|---|---|---|---|---|---|---|---|
| | | (copies) | | | Tumor size (cm) | | |
| | | 1 | 3 | 1 | 1 | 3 | 1 |
| | | month | months | year | month | months | year |
| Recurrence | Case1 | 60.9 | 201.4 | 1010.3 | N.D.* | N.D. | 1 |
| | Case5 | 10.6 | 50.8 | 898.9 | N.D. | N.D. | <1 |
| No | Case2 | 20.6 | 30.5 | 60.3 | N.D. | N.D. | N.D. |
| Recurrence | Case3 | 50.4 | 40.8 | 48.6 | N.D. | N.D. | N.D. |
| | Case4 | 60.9 | 89.6 | 88.3 | N.D. | N.D. | N.D. |

*N.D. stands for not detected.

### Example 16

### Testing Kit for Real Time PCR Measurement of Splice 1 of CST1 with TaqMan Probes

The kit includes the following: 1) PCR primers: 5′-tctcaccctcctctcctg-3′ (SEQ ID No.1) and 5′-ttatcctatcctcctccttgg-3′ (SEQ ID No.2). 2) Probe: 5′-FAM-ctccagctttgtgctctgcctct-TAMRA-3′ (SEQ ID No.3).

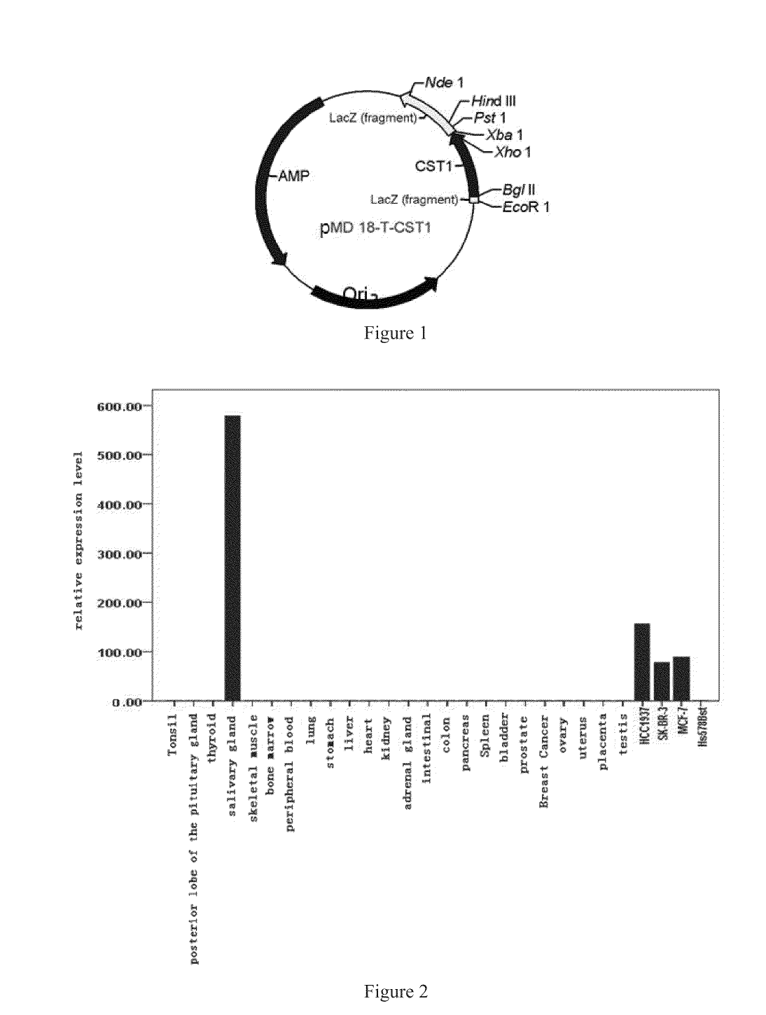

One or more of the following can be involved in the kit: RNA extraction reagents, reverse transcription reagents, deoxynucleotide triphosphates (dNTP), buffers, magnesium chloride solutions, DNA polymerase, recombinant plasmid standard with splice 1 of CST1 amplicon sequence (SEQ ID No.51) (as shown in FIG. 1. Primers are 5′-gggctccctgcctcgggctctcac-3′ (SEQ ID No.22) and 5′-acggtctgttgcctggctcttagt-3′ (SEQ ID No.23)), positive control (breast cancer cell line HCC1937) and negative control (breast tissue cell line Hs578Bst).

In a typical procedure, all the sample, controls and standard are amplified by PCR. The CP values of the standards versus concentrations of the standards are plotted and a calibration equation is acquired through data fitting. The gene quantification is realized by comparing the CP value with the calibration curve.

### Example 17

### Testing Kit for Real Time PCR Measurement of Splice 1 of CST1 with Fluorescent Dyes

At least one pair of the following primers should be included. 5′-tctcaccct-cctctcctg-3′ (SEQ ID No.1); 5′-ttatcctatcctcctccttgg-3′ (SEQ ID No.2). 5′-ccctgggagaacagaaggtcc-3′ (SEQ ID No.4); 5′-ggtggtggctggtgcgaat-3′ (SEQ ID No.5). 5′-cattcgcaccagccaccac-3′ (SEQ ID No.6); 5′-agaagcaa-gaaggaaggagggag-3′ (SEQ ID No.7). 5′-cagcgtgcccttcacttcg-3′ (SEQ ID No. 8); 5′-cggtctgttgcctggctctta-3′ (SEQ ID No.9). 5′-cattcgcaccagcca-ccac-3′ (SEQ ID No.10); 5′-cagggctatagaagcaagaaggaa-3′ (SEQ ID No.11). 5′-ggtacagcgtgcccttcacttc-3′ (SEQ ID No.12); 5′-cggtctgttgcctggctctta-3′ (SEQ ID No.13). 5′-gagaacagaaggtccctggtgaa-3′ (SEQ ID No.14); 5′-ggtggtggctggtgcgaat-3′ (SEQ ID No.15). 5′-tgggtacagcgtgcccttca-3′ (SEQ ID No.16); 5′-cggtctgttgcctggctctta-3′ (SEQ ID No.17). 5′-ccctgggagaacagaaggtcc-3′ (SEQ ID No.18); 5′-tggtggctggtgcgaatgg-3′ (SEQ ID No. 19). 5′-ttccctgggag-aacagaaggtcc-3′ (SEQ ID No.20); 5′-tggtgg-ctggtgcgaatgg-3′ (SEQ ID No.21).

One or more of the following can be involved in the kit: primers for internal marker β-actin 5′-aagatcattgctcctcctg-3′ (SEQ ID No.32) 5′-cgtcatactcctgcttgc-3′ (SEQ ID No.33), RNA extraction reagents, reverse transcription reagents, deoxynucleotide triphosphates (dNTP), buffers, magnesium chloride solutions, DNA polymerase and fluorescent dyes (such as SYBR Green).

### Example 18

### Test Kit for Expression of Splice 1 of CST1 Based on NASBA

The kit includes the following: 1) primers: 5′-aattctaatacgactca-ctatagggtctcaccctcctctcctg-3′ (SEQ ID No.34) and 5′-ttatcctatcctcctccttgg-3′ (SEQ ID No.2). 2) probe: 5′-FAM-ctccagctttgtgctctgcctct-Dabsyl-3′ (SEQ ID No.3) One or more of the following can be involved in the kit: RNA extraction reagents, reverse transcription reagents, fluorescent dyes for RNA (such as Ribo-Green), T4 RNA polymerase, RNase H, Avian myeloid leukemia virus (AMV) reverse transcriptase, ribonuleotide tirphosphate (NTP) and dNTP.

### Example 19

### Test Kit for Expression of Splice 1 of CST1 Based on TMA

The kit includes the following: 1) Primers: 5′-aattctaatacgactcactatagggtctcaccctcctctcctg-3′ (SEQ ID No.34) and 5′-ttatcctatcctcctccttgg-3′ (SEQ ID No.2). 2) probe: 5′-FAM-ctccagattgtgctctgcctct-Dabsyl-3′ (SEQ ID No.3).

One or more of the following can be involved in the kit: RNA extraction reagents, reverse transcription reagents, fluorescent dyes for RNA (such as Ribo-Green), T4 RNA polymerase, RNase H, Avian myeloid leukemia virus (AMV) reverse transcriptase, NTP and dNTP.

### Example 20

### Test Kit for Expression of Splice 1 of CST1 Based on LCR

The kit includes the 4 primers: 5′-agtatctgagtaccctgctgctcctgc-3′ (SEQ ID No.35), 5′-accctagctgtggccctggcctggag-3′ (SEQ ID No.36), 5′-catagactcatgggacgacg-3′ (SEQ ID No.37), 5′-acaccgggaccggacctc-3′ (SEQ ID No.38). All primers have heptane labeling on the 5′ termini.

One or more of the following can be involved in the kit: RNA extraction reagents, reverse transcription reagents, T4 DNA ligase and dNTP.

### Example 21

### Test Kit for Expression of Splice 1 of CST1 Based on tSDA

The kit includes the following: 1) Primer for CST1 B1: 5′-tgggtacagc-gtgcccttcactt-3′ (SEQ ID No.39), primer for CST1 S1: 5′-ccgctcgagtacagcgtgcccttcacttcgc-3′ (SEQ ID No.40), primer for CST1 B2: 5′-caacggtctgttgcctggctctta-3′ (SEQ ID No.41) and primer for CST1 S2: 5′-gacctcgaggttgcctggctcttagtacccg-3′ (SEQ ID No.42). 2) probe: 5′-gtgctcgagtcagcgagtataacaagg-ccaccaaagatgactac-3′ (SEQ ID No.3) with ³²P labeling on the 5′ terminus.

One or more of the following can be involved in the kit: RNA extraction reagents, reverse transcription reagents, dCTPaS, dATP, dGTP, dTTP, Bsobl enzyme, exo-Bca enzyme.

( [symbol] ) Protein

Recombinant cystatin SN, rabbit ant-cystatin SN antibody were purchased from NOVUS Biologicals. Mouse anti-human cystatin SN monoclonal antibody (with specific recognition to the sequence shown in SEQ ID No. 54) was purchased from R&D. TMB oxidase substrate, including TMB solution A and peroxidase solution B, was purchased from Kirkegaard and Perry Laboratories Inc.

A typical procedure for serum preparation is described as following: the whole blood samples are stored under ambient temperature for 2 hours or overnight under 4° C., after which the sample is centrifuged for 20 minutes (1000 g). Supernatant is collected for testing or storage under −20° C. or −80° C. Repeated freezing and thawing should be avoided. Plasma samples are prepared by the following protocol: blood samples added with anticoagulant such as EDTA or heparin are centrifuged for 15 minutes (2-8° C., 1000 g) and the supernatant is collected for testing or storage under −20° C. or −80° C. Repeated freezing and thawing should be avoided. The serum and plasma samples are diluted 10 times using PBS buffer (0.1 M, pH 7.0-7.2) before testing.

### Example 22

### Cystatin Expression in Breast Cancer Cell Lines and Normal Human Serums

Breast cancer cell lines HCC1937 (Lanes 1-2) and MCF-7 (Lanes 3-4) with high expression of CST1 mRNA and normal human serums 51 (Lanes 5-6) and S2 (Lanes 7-8) were analyzed by SDS-PAGE (15%). The proteins were transferred to nitrocellulose membrane and incubated with coating solution (5% skimmed milk powder and 0.1% TWEEN-20), after which the membrane was incubated overnight with polyclonal anti-cystatin SN antibody. The membrane was then washed with PBS with 0.1% TWEEN-20 for three times, incubated with horserumsddish peroxidase (HRP) labeled goat-anti-rabbit IgG solution for an hour under 37° C., washed with PBS with 0.1% TWEEN-20 for four times, washed with PBS once and treated with TMB peroxidase indicator for colorimetric detection. β-actin was used as internal reference. As shown in FIG. 17, a protein of 16 kDa was observed on Lanes 1-4, while it is not significant in Lanes 5-8. It is concluded that cystatin SN expression is abnormally high in breast cancer cells.

### Example 23

### Cystatin SN Expression in Serums of Breast Cancer Patients and Healthy People

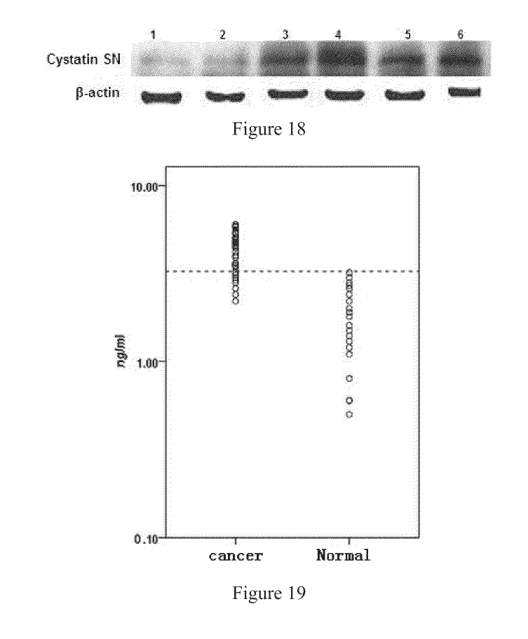

Serums of healthy people (Lanes 1-2) and breast cancer patients (Lanes 3-6) were analyzed by SDS-PAGE and immunoblotting as described in Example 22. As shown in FIG. 18, cystatin SN detected in Lanes 3-6 was much higher than in Lanes 1-2, indicating that cystatin SN expression is abnormally high in breast cancer patients serums.

### Example 24

### Cystatin SN Expression in Serums of Breast Cancer Patients and Healthy People Measured by Competitive ELISA

The ELISA plate was coated by 5 μg/mL cystatin SN solution and backfilled by 3% BSA. A serum sample was mixed with cystatin SN monoclonal antibody (1:2000) and the mixture was incubated in the plate for 1 hour (37° C.). The ELISA plate was washed with TBS buffer (154 mM NaCl, 10 mM Tris-HCl, pH 7.5) and incubated by HRP-labeled goat-anti-rabbit IgG (0.08 μg/mL) for an hour under 37° C. The plate was washed again by TBS. Finally the plate was treated by o-phenylenediamine for quantitative detections by a microplate reader. 20 serum samples from healthy people and 30 serum samples from breast cancer patients were tested. As shown in FIG. 19, cystatin SN concentration median for normal serum is 1.7 ng/mL, while it reaches 4.1 ng/mL for breast cancer patients. Breast cancer samples are distinguishable if 3.25 ng/mL is the cutoff value.

### Example 25

### Cystatin SN Expression in Serums of Breast Cancer Patients and Healthy People Measured by Double-Antibody Sandwich ELISA

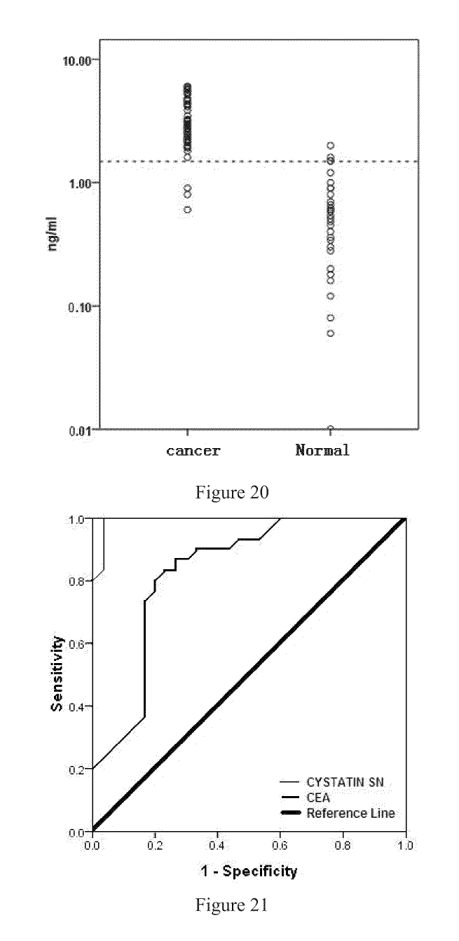

The ELISA plate was coated by 5 μg/mL mouse-anti-human cystatin SN monoclonal antibody and backfilled by 3% BSA solution. A serum sample was incubated with the plate for 1 hour (37° C.) and washed by TBS buffer. Biotin-labeled rabbit-anti-cystatin SN polyclonal antibody (1:1000) was incubated in the plate. The plate was washed by TBS and added with streptavidin-peroxidase substrate conjugate, which was incubated for another hour and washed by TBS buffer. TMB was added in the plate and the colorimetric signal was measured by a microplate reader. 20 serum samples from healthy people and 30 serum samples from breast cancer patients were tested. As shown in FIG. 20, cystatin SN concentration median for normal serum is 0.525 ng/mL, while it reaches 2.99 ng/mL for breast cancer patients. Breast cancer samples are distinguishable if 1.48 ng/mL is the cutoff value.

### Example 26

### Sensitivity and Specificity Comparison of Cystatin SN and CEA in Serum as a Marker for Breast Cancer

Cystatin SN expression was measured following the protocol measured in Example 25. CEA was measured using commercially available testing kits. 30 normal serum samples and 30 serum samples from breast cancer patients were tested and the results are summarized in FIG. 21. AUC values are 0.994 and 0.833 for cystatin SN and CEA respectively, which indicates that cystatin SN is advantageous as a breast cancer marker than CEA if sensitivity and specificity are concerned.

### Example 27

### Testing Kit for Quantification of Cystatin SN (Competitive ELISA) 1. Composition of Kit

The following should be included: cystatin SN, mouse-anti-human cystatin SN monoclonal antibody, HRP-labeled goat-anti-rabbit IgG, enzymatic substrate (o-phenylenediamine)

The kit might include the following: backfilling solution (3% BSA), TBS buffer (154 mM NaCl and 10 mM Tris-HCl, pH 7.5)

2. Guideline

Coat the ELISA plate with 5 mg/mL cystatin SN. The plate is then backfilled by 3% BSA solution. Serum sample and anti-cystatin SN monoclonal antibody are mixed (1:2000) and incubated for 1 hour (37° C.) in the plate. The plate is washed by PBS and added with HRP labeled goat-anti-rabbit IgG (0.08 μg/mL) with 1 hour incubation (37° C.). The plate was added 0.4 mg/mL o-phenylenediamine and microplate reader is used for measurment.

### Example 28

### Testing Kit for Quantification of Cystatin SN (Double-Antibody Sandwich ELISA)

1. Composition of Kit

The following should be included: cystatin SN standard (make a series of solutions using PBST with 1% BSA. The concentrations are 10, 5, 2.5, 1, 0.5 and 0.25 ng/mL for instance), mouse-anti-human cystatin SN monoclonal antibody, biotin-labeled rabbit-anti-cystatin SN polyclonal antibody, streptavidin-substrate conjugate, TMB peroxidase substrate.

The kit might include the following: coating solution (0.05 M NaHCO₃, pH 9.0), backfilling solution (3% BSA), PBST buffer with 1% BSA, PBST buffer (PBS with 0.05% TWEEN-20) and stop solution (2 N H₂SO₄).

2. Guideline

Dissolve mouse-anti-human cystatin SN monoclonal antibody in the dilution buffer with a concentration of 5 μg/mL which are added in the ELISA plate. Incubate the plate overnight under 4° C. Discard the residue liquid and wash the plate for three times using PBST buffer for 9 minutes in total. Add 200 μL backfilling solution in the holes of the plate and incubate the plate overnight under 4° C. or for one hour under 37° C. Discard the residue liquid and wash the plate for three times using PBST buffer for 9 minutes in total. Assign sample holes for blank, standard, sample. Add 100 μL PBS, cystatin SN standard solution and serum/plasma samples in the blank, standard and sample hole respectively. Incubate the plate for 2 hours (37° C.). Discard the residue liquid. Add 100 μL biotin-labeled rabbit-anti-cystatin SN polyclonal antibody solution (diluted by PBST-BSA buffer with rato of 1:200). Incubate the plate for 1 hour (37° C.) and discard the residue liquid in the holes. Wash the plate by PBST buffer for 3 times (9 minutes in total). Add streptavidin-substrate conjugate in the holes and incubate the plate for 1 hour (37° C.). Wash the plate for five cycles with 1-2 minutes for each cycle. Add 50 μL peroxidase substrate solution in the holes and keep the plate in dark. Stop the reaction in around 15 minutes when the standard holes shows gradient blue color by adding 50 μL stop solution (with color change from blue to yellow). Test the OD values using a microplate reader (405 nm). Plot OD's of standards v.s. concentration to gain the calibration curve and R² value (if R² is higher than 0.95, then the calibration curve is validated). Cystatin SN concentration is calculated by the OD value of the sample hole and the calibration equation.

### Example 29

### Application of Cystatin SN for Breast Cancer pTNM Staging

The serum samples of 80 breast cancer patients (20 in Stage T, 30 in stage N and 30 in stage M) were tested using the protocol/kit described in Example 28. As summarized in Table 4, cystatin SN expression increases with the continuation of the cancer development. The result indicates the application of cystatin SN in the pTMN staging.

TABLE 4
Cystatin SN levels in serums with
breast cancer with various stages

| | Median of Serum |
|---|---|
| | Cystatin SN Level (ng/mL) |
| Stage T (20 cases) | 0.96 |
| State N (30 cases) | 1.48 |
| State M (30 cases) | 3.34 |

### Example 30

### Metastasis Detection by Cystatin SN Level

20 serum samples from breast cancer patients (no metastasis) and 30 serum samples from breast cancers with metastasis were tested via the protocol mentioned in Example 28. As shown in Table 5, the cystatin SN levels from metastatic breast cancer patients are higher than those patients without metastasis, evidencing that cystatin SN is a marker for breast cancer metastasis.

TABLE 5
Cystatin SN levels in breast cancer patients

| | Median of Cystatin |
|---|---|
| | SN Level (ng/mL) |
| Breast cancer without | 1.06 |
| metastasis (20 cases) | |
| Metastatic breast cancer | 3.29 |
| (30 cases) | |

### Example 31

### Cystatin SN for the Evaluation of the Effect of Chemotherapy (CT)-Endocrine Therapy Combination

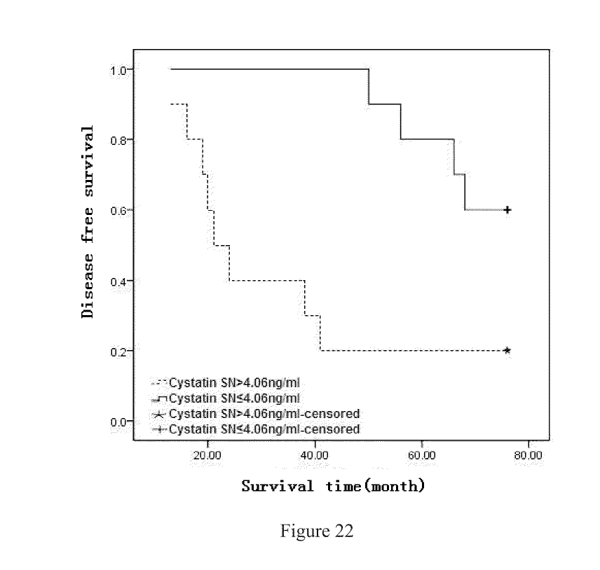

2885 breast cancer patients (NO or Ni) were treated with AC (Doxorubicin+cyclophosphamide) or AT (Doxorubicin+paclitaxel). Endocrine therapy was opted based on hormone receptor (HR) levels. Cystatin SN was measured using the protocol in Example 28. 776 cases were verified for data analysis, among which the median cystatin SN concentration is 4.06 ng/mL. The prognosis-free survival (PFS) was recorded in the following 76 months. As shown in FIG. 22, the patients with higher cystatin SN expression than the median have PFS of 20%; the others have PFS around 60%. The results evidence that cystatin SN is an index for the evacuation of the efficacy of the therapies.

The examples above discussed the invention in great detail. It should be noted that these examples can be combined to meet the requirements for real practice. Replacements, modifications and changes are necessary and straight forward to researchers in this area, and thus should be part of this invention.

1. The applications of CST1 gene including its splice variants, cystatin SN which is coded by CST1 gene and its splice variants, and the epitope peptide of cystatin SN for the prediction and diagnosis of human breast cancers. The sequence of CST1 gene and its splice variants are presented in SEQ ID No.48; the sequence of cystatin SN is presented in SEQ ID No. 52.

2. A method according to claim 1, wherein the sequence of the epitope peptide of cystatin SN is shown in SEQ ID No.54.

3. A method according to claim 1, wherein breast cancers to be predicted and diagnosed include original and metastatic human breast cancers. Analysis for the susceptibility for breast cancer, the treatment, medicine, efficacy and evaluation of the treatments for original or metastatic breast cancer, and risk assessment for breast cancer or its metastasis are included in this claim.

4. The applications of the “Capturers” for CST1 gene, its splice variants and cystatin SN in manufacturing diagnostic reagents, kits and chips for breast cancer predictions and diagnoses. Captures of CST1 gene and its splices include primers or probes that specifically recognize CST1 or its cDNA. Cystatin SN Capturer includes antibodies for cystatin SN. The sequence of CST1 gene and its splice variants are presented in SEQ ID No.48; the sequence of cystatin SN is presented in SEQ ID No. 52.

5. A method according to claim 4, wherein the primer for CST1 gene and its splice variants should be selected from and include at least one the sequences shown in SEQ ID No.1-2, 4-21, 34 and 39-42.

6. A method according to claim 5, wherein the corresponding probes for primers with sequences shown in SEQ ID No.1-2 have the sequence shown in SEQ ID No.3.

7. Applications of the diagnostic kits which include capturers for CST1 gene, its splices and the protein cystatin SN which is coded by CST1 gene and its splices, in the manufacturing of the diagnostic kits for breast cancer predictions and diagnoses. Capturers of CST1 gene and its splice variants include primers or probes that specifically recognize CST1 or its cDNA. Cystatin SN Capturer includes antibodies for cystatin SN. The sequences of CST1 gene and its splice variants are presented in SEQ ID No.48; the sequence of cystatin SN is presented in SEQ ID No. 52.

8. A method according to claim 7, wherein the primers for CST1 gene and its splice variants should be selected from the sequences shown in SEQ ID No.1-2, 4-21, 34 and 39-42, and wherein the probe for CST1 gene and its splice variants should be selected from one of the sequences shown in SEQ ID No 3, 35-38 and 43.

9. A method according to claim 8, wherein the primers have sequences presented in SEQ ID No. 1-2 and the corresponding probe has a sequence shown in SEQ ID No.3.

10. Chips with CST1 gene (including its splices) capturers immobilizations in the applications for breast cancer predictions and diagnoses. The capturers herein includes primers or probes that specifically recognize CST1, splices of CST1 and their cDNA. The sequences of CST1 gene and its splice variants are presented in SEQ ID No.48.

11. A method according to claim 10, wherein the probes should be selected from the sequences of SEQ ID No. 3, 35-38 and 43.

12. A method according to claim 11, wherein the probes should have the sequence shown in SEQ ID No.3.

13. Predictive and diagnostic kits using capturers for CST1 gene and its splice variants capturer, wherein CST1 gene and its splice variants Capturer includes primers or probes that specifically recognize CST1 or its cDNA. The sequences for the primers are presented in SEQ ID No.1-2 and the sequence for the probe is shown in SEQ ID No.3, with fluorophore labeling on 5′ terminus and fluorescent quencher labeling on 3′ terminus.

14. A predictive and diagnostic kit according to claim 13, wherein the kit includes standards of recombinant plasmid of CST1 gene and its splices (amplicon), whose sequence is presented in SEQ ID No.51.

15. A predictive and diagnostic kit according to claim 14, wherein the kit includes breast cancer cell line HCC 1937 as a positive control and normal breast cell line Hs578Bst as a negative control.

16. A predictive and diagnostic kit for breast cancer, wherein the capturer of CST1 gene and its splices is the specific primers and probes for the cDNA of CST1 gene and its splices. The sequences of the primers are presented in SEQ ID No. 34 and 2. The sequences of the probe is presented in SEQ ID No. 3, with fluorophore labeling on 5′ terminus and fluorescent quencher labeling on 3′ terminus.

17. A predictive and diagnostic kit for breast cancer which includes the capturers of CST1 gene and its splice variants, wherein the capturers herein are the primer and probe for the cDNA of CST1 gene and its splice variants. The sequences of the primers are presented in SEQ ID No. 39-44. The sequences of the probe is presented in SEQ ID No. 43, with radioactive labeling on 5′ terminus.

18. A predictive and diagnostic kit for breast cancer which includes the capturers of CST1 gene and its splice variants, wherein the capturers herein are the primers for the cDNA of CST1 gene and its splice variants, whose sequences are presented in SEQ ID No.1-2, or SEQ ID No.4-5, or SEQ ID No.6-7, or SEQ ID No.8-9, or SEQ ID No.10-11, or SEQ ID No.12-13, or SEQ ID No.14-15, or SEQ ID No.16-17, or SEQ ID No.18-19, or SEQ ID No.20-21.

19. A predictive and diagnostic kit for breast cancer which includes the CST1 gene and its splice variants capturer, wherein the CST1 gene and its splice variants capturer is the specific probe for the cDNA of CST1 gene and its splice variants. The sequences of the probes are presented in SEQ ID No.35-38, with hapten labeling on 5′ terminus.

20. A predictive and diagnostic kit for breast cancer which includes the capturers for CST1 coded protein cystatin SN, wherein the cystatin SN capturer is the monocolonal antibody of cystatin SN. The kit includes cystatin SN antigen, secondary antibody for immunoassay and the enzymatic substrate.

21. A predictive and diagnostic kit for breast cancer which includes the capturer for CST1 coded protein cystatin SN, wherein the capturer for cystatin SN is the monoclonal antibody of cystatin SN and biotin labeled anti-cystatin SN polyclonal antibody. The kit includes cystatin SN standard, streptavidin-enzyme conjugate and enzymatic substrate.

22. Any methods for the predictions and diagnoses of breast cancer, or any kits for predictions and diagnoses of breast cancer according to claim 13, wherein the measured level or expression of CST1 gene and its splice variants or proteins coded by CST1 gene and its splice variants such as cystatin SN in the samples, is compared with the cutoff value. The conclusion of the test is positive if what is measured is higher than the cutoff value. The cutoff values are obtained via comparison with statistical significance of the levels or expression of CST1 gene and its splice variants, and proteins coded by CST1 gene and its splice variants cystatin SN in the tissues or body fluid of healthy people and breast cancer patients. Samples include tissues from surgery or biopsy, lymph node tissues, marrow, serum, plasma, whole blood, fractions of blood and urine.


SEQUENCE LISTING (54 sequences)

SEQ ID NO: 1 - DNA, 18 bp, Artificial Sequence
  Oligonucleotide
tctcaccctcctctcctg

SEQ ID NO: 2 - DNA, 21 bp, Artificial Sequence
  Oligonucleotide
ttatcctatcctcctccttgg

SEQ ID NO: 3 - DNA, 23 bp, Artificial Sequence
  Oligonucleotide
ctccagctttgtgctctgcctct

SEQ ID NO: 4 - DNA, 21 bp, Artificial Sequence
  Oligonucleotide
ccctgggagaacagaaggtcc

SEQ ID NO: 5 - DNA, 19 bp, Artificial Sequence
  Oligonucleotide
ggtggtggctggtgcgaat

SEQ ID NO: 6 - DNA, 19 bp, Artificial Sequence
  Oligonucleotide
cattcgcaccagccaccac

SEQ ID NO: 7 - DNA, 23 bp, Artificial Sequence
  Oligonucleotide
agaagcaagaaggaaggagggag

SEQ ID NO: 8 - DNA, 19 bp, Artificial Sequence
  Oligonucleotide
cagcgtgcccttcacttcg

SEQ ID NO: 9 - DNA, 21 bp, Artificial Sequence
  Oligonucleotide
cggtctgttgcctggctctta

SEQ ID NO: 10 - DNA, 19 bp, Artificial Sequence
  Oligonucleotide
cattcgcaccagccaccac

SEQ ID NO: 11 - DNA, 24 bp, Artificial Sequence
  Oligonucleotide
cagggctatagaagcaagaaggaa

SEQ ID NO: 12 - DNA, 22 bp, Artificial Sequence
  Oligonucleotide
ggtacagcgtgcccttcacttc

SEQ ID NO: 13 - DNA, 21 bp, Artificial Sequence
  Oligonucleotide
cggtctgttgcctggctctta

SEQ ID NO: 14 - DNA, 23 bp, Artificial Sequence
  Oligonucleotide
gagaacagaaggtccctggtgaa

SEQ ID NO: 15 - DNA, 19 bp, Artificial Sequence
  Oligonucleotide
ggtggtggctggtgcgaat

SEQ ID NO: 16 - DNA, 20 bp, Artificial Sequence
  Oligonucleotide
tgggtacagcgtgcccttca

SEQ ID NO: 17 - DNA, 21 bp, Artificial Sequence
  Oligonucleotide
cggtctgttgcctggctctta

SEQ ID NO: 18 - DNA, 21 bp, Artificial Sequence
  Oligonucleotide
ccctgggagaacagaaggtcc

SEQ ID NO: 19 - DNA, 19 bp, Artificial Sequence
  Oligonucleotide
tggtggctggtgcgaatgg

SEQ ID NO: 20 - DNA, 23 bp, Artificial Sequence
  Oligonucleotide
ttccctgggagaacagaaggtcc

SEQ ID NO: 21 - DNA, 19 bp, Artificial Sequence
  Oligonucleotide
tggtggctggtgcgaatgg

SEQ ID NO: 22 - DNA, 24 bp, Artificial Sequence
  Oligonucleotide
gggctccctgcctcgggctctcac

SEQ ID NO: 23 - DNA, 24 bp, Artificial Sequence
  Oligonucleotide
acggtctgttgcctggctcttagt

SEQ ID NO: 24 - DNA, 19 bp, Artificial Sequence
  Oligonucleotide
agtcccagcccaacttgga

SEQ ID NO: 25 - DNA, 24 bp, Artificial Sequence
  Oligonucleotide
gggaacttcgtagatctggaaaga

SEQ ID NO: 26 - DNA, 20 bp, Artificial Sequence
  Oligonucleotide
gcactggggaagagaggcat

SEQ ID NO: 27 - DNA, 23 bp, Artificial Sequence
  Oligonucleotide
aggagcagcagggtactcagata

SEQ ID NO: 28 - DNA, 20 bp, Artificial Sequence
  Oligonucleotide
cagaagaaacagttgtgctc

SEQ ID NO: 29 - DNA, 18 bp, Artificial Sequence
  Oligonucleotide
ggagtaggaggtggtcag

SEQ ID NO: 30 - DNA, 20 bp, Artificial Sequence
  Oligonucleotide
gctctcaccctcctctcctg

SEQ ID NO: 31 - DNA, 20 bp, Artificial Sequence
  Oligonucleotide
tatcctattctcctccttgg

SEQ ID NO: 32 - DNA, 19 bp, Artificial Sequence
  Oligonucleotide
aagatcattgctcctcctg

SEQ ID NO: 33 - DNA, 18 bp, Artificial Sequence
  Oligonucleotide
cgtcatactcctgcttgc

SEQ ID NO: 34 - DNA, 43 bp, Artificial Sequence
  Oligonucleotide
aattctaatacgactcactatagggtctcaccctcctctcctg

SEQ ID NO: 35 - DNA, 27 bp, Artificial Sequence
  Oligonucleotide
agtatctgagtaccctgctgctcctgc

SEQ ID NO: 36 - DNA, 26 bp, Artificial Sequence
  Oligonucleotide
accctagctgtggccctggcctggag

SEQ ID NO: 37 - DNA, 20 bp, Artificial Sequence
  Oligonucleotide
catagactcatgggacgacg

SEQ ID NO: 38 - DNA, 18 bp, Artificial Sequence
  Oligonucleotide
acaccgggaccggacctc

SEQ ID NO: 39 - DNA, 23 bp, Artificial Sequence
  Oligonucleotide
tgggtacagcgtgcccttcactt

SEQ ID NO: 40 - DNA, 31 bp, Artificial Sequence
  Oligonucleotide
ccgctcgagtacagcgtgcccttcacttcgc

SEQ ID NO: 41 - DNA, 24 bp, Artificial Sequence
  Oligonucleotide
caacggtctgttgcctggctctta

SEQ ID NO: 42 - DNA, 31 bp, Artificial Sequence
  Oligonucleotide
gacctcgaggttgcctggctcttagtacccg

SEQ ID NO: 43 - DNA, 44 bp, Artificial Sequence
  Oligonucleotide
gtgctcgagtcagcgagtataacaaggccaccaaagatgactac

SEQ ID NO: 44 - DNA, 782 bp, Homo sapiens
gggctccctgcctcgggctctcaccctcctctcctgcagctccagctttgtgctctgcctctgaggagaccatggcccagtatctgagtaccctgctgctcctgctggccaccctagctgtggccctggcctggagccccaaggaggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggagaacagaaggtccctggtgaaatccaggtgtcaagaatcctagggatctgtgccaggccattcgcaccagccaccacccactcccaccccctgtagtgctcccacccctggactggtggcccccaccctgcgggaggcctccccatgtgcctgcgccaagagacagacagagaaggctgcaggagtcctttgttgctcagcagggcgctctgccctccctccttccttcttgcttctaatagccctggtacatggtacacacccccccacctcctgcaattaaacagtagcatcgcctccctctgaaaaaaaaaaaaaaaaaaaaaaa

SEQ ID NO: 45 - DNA, 299 bp, Homo sapiens
gggctccctgcctcgggctctcaccctcctctcctgcagctccagctttgtgctctgcctctgaggagaccatggcccagtatctgagtaccctgctgctcctgctggccaccctagctgtggccctggcctggagccccaaggaggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacag

SEQ ID NO: 46 - DNA, 114 bp, Homo sapiens
accgttgggggggtgaattacttcttcgacgtagaggtgggccgcaccatatgtaccaagtcccagcccaacttggacacctgtgccttccatgaacagccagaactgcagaag

SEQ ID NO: 47 - DNA, 347 bp, Homo sapiens
aaacagttgtgctctttcgagatctacgaagttccctgggagaacagaaggtccctggtgaaatccaggtgtcaagaatcctagggatctgtgccaggccattcgcaccagccaccacccactcccaccccctgtagtgctcccacccctggactggtggcccccaccctgcgggaggcctccccatgtgcctgcgccaagagacagacagagaaggctgcaggagtcctttgttgctcagcagggcgctctgccctccctccttccttcttgcttctaatagccctggtacatggtacacacccccccacctcctgcaattaaacagtagcatcgcctccctctga

SEQ ID NO: 48 - DNA, 760 bp, Homo sapiens
gggctccctgcctcgggctctcaccctcctctcctgcagctccagctttgtgctctgcctctgaggagaccatggcccagtatctgagtaccctgctgctcctgctggccaccctagctgtggccctggcctggagccccaaggaggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggagaacagaaggtccctggtgaaatccaggtgtcaagaatcctagggatctgtgccaggccattcgcaccagccaccacccactcccaccccctgtagtgctcccacccctggactggtggcccccaccctgcgggaggcctccccatgtgcctgcgccaagagacagacagagaaggctgcaggagtcctttgttgctcagcagggcgctctgccctccctccttccttcttgcttctaatagccctggtacatggtacacacccccccacctcctgcaattaaacagtagcatcgcctccctctga

SEQ ID NO: 49 - DNA, 655 bp, Homo sapiens
caggaagtcacatagctttcacagtcaaaggaccagtgtcacctctgtggagacctgggtgactcaagcttgggagcactggggaagagaggcatggctcggggaggctgcactccagctttgtgctctgcctctgaggagaccatggcccagtatctgagtaccctgctgctcctgctggccaccctagctgtggccctggcctggagccccaaggaggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggagaacagaaggtccctggtgaaatccaggtgtcaagaatcctagggatctgtgccaggccattcgcaccagccaccacccactcccaccccctgtagtgctcccacccctggactggtggcccccaccc

SEQ ID NO: 50 - DNA, 141 bp, Homo sapiens
tctcaccctcctctcctgcagctccagctttgtgctctgcctctgaggagaccatggcccagtatctgagtaccctgctgctcctgctggccaccctagctgtggccctggcctggagccccaaggaggaggataggataa

SEQ ID NO: 51 - DNA, 304 bp, Homo sapiens
gggctccctgcctcgggctctcaccctcctctcctgcagctccagctttgtgctctgcctctgaggagaccatggcccagtatctgagtaccctgctgctcctgctggccaccctagctgtggccctggcctggagccccaaggaggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgt

SEQ ID NO: 52 - protein, 141 aa, Homo sapiens
MAQHLSTLLLLLATLAVALAWSPKEEDRIIPGGIYNADLNDEWVQRALHFAISEYNKATKDDYYRRPLRVLRARQQTVGGVNYFFDVEVGRTICTKSQPNLDTCAFHEQPELQKKQLCSFEIYEVPWENRRSLVKSRCQES

SEQ ID NO: 53 - DNA, 426 bp, Homo sapiens
atggcccagtatctgagtaccctgctgctcctgctggccaccctagctgtggccctggcctggagccccaaggaggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggagaacagaaggtccctggtgaaatccaggtgtcaagaatcctag

SEQ ID NO: 54 - protein, 121 aa, Homo sapiens
WSPKEEDRIIPGGIYNADLNDEWVQRALHFAISEYNKATKDDYYRRPLRVLRARQQTVGGVNYFFDVEVGRTICTKSQPNLDTCAFHEQPELQKKQLCSFEIYEVPWENRRSLVKSRCQES